(19) United States
(12) Patent Application Publication
(10) Pub. No.: US 2024/0230482 A1
(43) Pub. Date: Jul. 11, 2024

(54) METHOD FOR EXTRACTING AND DETECTING REAL ODOROUS MOLECULES OF INTEREST AND EXTRACTION DEVICE IMPLEMENTING SAID METHOD

(71) Applicant: Biodesiv Sarl, Neuchâtel (CH)

(72) Inventors: Quentin Courrier, Neuchâtel (CH); Grégory Herin, Neuchâtel (CH)

(21) Appl. No.: 18/288,331

(22) PCT Filed: Apr. 21, 2022
(86) PCT No.: PCT/EP2022/060634

§ 371 Date: Oct. 25, 2023

(30) Foreign Application Priority Data
Apr. 26, 2021 (FR) FR2104307

(51) Int. Cl. G01N 1/22

(52) U.S. Cl. CPC G01N 1/2273; G01N 1/2214; G01N 1/2226

(57) ABSTRACT

The present invention relates to a dynamic method for detecting actual odorous molecules of interest contained in the atmosphere or in direct proximity to the atmosphere present in an enclosure comprising the following steps: a) Sucking up a sample of the atmosphere by means of an extraction device; b) Making said sample circulate/pass throughout and/or over a collection device capable of adsorbing the odorous molecules, said device being in the form of a hollow tube open at each of its ends; and c) Having said device analyzed by a detection animal, and an extraction device for implementing the method.

### FIELD OF THE INVENTION

The present invention relates to the field of control and safety of enclosures of any size through the collection of actual odorous molecules of interest. Thus, the invention relates to a method and a device for the collection, storage, transport and analysis of actual odorous molecules of interest contained in the atmosphere in or proximate to an enclosure from a sample of said atmosphere.

### PRIOR ART

The ability to detect odors associated with hazards or pathologies has been known and exploited for a very long time. Thus, detection animals are used in different fields such as health, the environment, detection and repression and even archeology. Among the detection animals, the dog is the most commonly used since on average, each dog inhales and exhales approximately five times per second and thus captures different scents which are instantly decoded by some 200 million receptor cells present in its nose. By comparison, humans only have 5 million on average, which makes the dog's olfactory system approximately 40 times more sensitive, and therefore more effective, than that of humans.

Among the most common applications of dogs' olfactory sense in the service of humans, one could mention the search for explosives, narcotics or human remains, tracking or detection of diseases.

It is also possible to detect odors by means of different machines capable of separating, identifying or quantifying the molecules that compose the odor. The most common technique is gas chromatography coupled with a mass spectrometer. For this purpose, it is necessary to collect the odor in order to introduce it into the measuring device, on a small support compatible with the equipment. The current sensitivity of these techniques remains approximately 1,000 times lower than that of some animals, in particular dogs. It also appears necessary to be able to concentrate the odor when it is collected in order to be able to analyze it.

Particularly in the area of law enforcement, not all freight cargo/goods in circulation can be controlled. For example, in European ports, less than 5% of containers are checked for possible current or past presence of products of interest. Nonetheless, these checks can be long and costly, which is the reason for their low frequency. On the contrary, the United States has made screening of all air cargo mandatory since the beginning of 2021.

Some checks are performed by an X-ray scanner, but the definition of the scanner does not make it possible to detect small products of interest scattered throughout the cargo. With the exception of a few easily recognizable products, like cigarettes or weapons, X-ray scanners do not give accurate information on the composition of the scanned items, in particular on their hazardous or illicit nature. Investments in these tools are also very high and require an electrical connection, thereby limiting use thereof to some areas.

At the same time, another type of control consists of visual inspections/manual searches assisted or not by detection dogs trained to recognize the odors of the products of interest. In this case, the searches are often partial as they are limited to areas easily accessible by humans or dogs. A complete inspection requires unpacking and then repacking the goods, the duration of inspection is then very significant as is the risk of injuring the agent in charge of the inspection, the dog or of damaging the goods.

Microparticle suction/filtration control systems provide a solution to these problems. Nonetheless, these systems are still very limited in terms of performance. Indeed, they function only if they are capable of collecting enough microparticles in the ambient air. However, some products to be controlled do not produce microparticles or are packaged in such a way as to prevent dissemination thereof. In addition, these systems become ineffective when the cargo contains a large amount of so-called “contaminating” particles, such as dust particles or powdery materials, which might clog the filter present on the suction/filtration device. Moreover, to ensure suction of a large volume of air in the shortest possible time, the filter should have the lowest possible pressure drop. The pressure drop is directly related to the porosity of the filter. Hence, to obtain a low pressure drop, a coarse filter (large mesh size) should be used, which retains a small amount of small-size particles or not at all and leads to poor detection. In the particular case of a cargo containing a lot of particles (for example: flour, dust, etc.) the filter can quickly become clogged and then becomes ineffective. What is more, the filters can retain parasites and their eggs or bacteria and viruses which would be present in the cargo and which would expose the detection dog to an infectious risk. Finally, the dimension of particle filters is also not compatible with devices for separation, detection or quantification of the molecules composing the odors.

It follows from the foregoing that it is essential to be able to perform cargo checks as quickly as possible, regardless of the nature of the cargo and with a high level of reliability, thereby increasing the number of seizures, while avoiding risks for the agents and dogs responsible for controlling goods, while limiting the degradation of the goods and having the possibility, where appropriate, to detect composing the molecules of an odor by means of separation, identification or quantification equipment

At the same time, animals, in particular dogs, are commonly used to detect the presence of hazardous organisms in buildings such as hotels, private homes and offices. For example, these organisms are bedbugs or some molds that are difficult to detect by humans. Detection animals, in particular dogs, must sometimes intervene in cluttered places (cellars, storage units, home of a person suffering from Diogenes syndrome, etc.), known to be reservoirs for colonies of insects or insects, mushrooms.

Current solutions for inspecting places potentially contaminated by unwanted organisms consist of having the dog smell every corner of the considered room. If the latter is cluttered, this forces the dog to smell the room from afar (loss of detection efficiency), to move the objects one-by-one by operators (risk of human contamination, injury and/or or object damage) or to make the dog move over the objects (risk of injury to the dog). The filtration collection device is currently not used due to its bulky nature and the need to have a nearby source of electrical current (electrical network or generator set).

It arises from the foregoing that it is essential to be able to analyze these buildings with analytical equipment or a detection animal, without any risk for the agent responsible for the control and the detection animal, with a view to providing them with the appropriate treatment.

As regards nosocomial diseases, these are infections contracted in a health establishment. It is estimated that in 2050, these infections could cause the deaths of 10 million patients per year and induce a treatment cost of around 100,000 billion dollars. Procedures exist to disinfect sensitive places. Nonetheless, the disinfection is never 100% effective and the overuse of antiseptic products leads to the development of resistance in bacteria and viruses to the action of antiseptic products. In addition, the introduction of measuring devices or detection animals, such as a dog, into an aseptic room increases the risk of re-contamination and even over-contamination of the room.

Consequently, there is a need to detect the potential presence of pathogens in these rooms without inducing contamination thereof.

The documents US 2008/250878 A1 and FR 2 956 211 A describe collection devices in the form of planar elements, arranged perpendicular to the circulation of the sucked air and through which the flow of sucked air is forced. The exchange surface between the sucked air and the collection device is limited and it is necessary to use a powerful suction means to force the air through these collection devices. Furthermore, in an atmosphere loaded with dust or suspended particles, clogging problems are frequently encountered.

The document FR 3 088 429 A1 describes a collection device in the form of a solid cylinder or a planar element having the same drawbacks as hereinabove. The solid nature of these elements leads to significant pressure drops making it necessary to equip the device with a powerful suction means as well as an air recirculation system so that the amount of absorbed odorous molecules is sufficient. The recirculation system presents a risk of pollution of the sorbent by odors/particles released by the materials of the recirculation system themselves or by remains of a previous collection. Cleaning is complex, expensive and with no guarantee of results.

It arises from the foregoing that there is an obvious need to put in place a detection device which is effective, reliable, rapid, which adapts to different areas of use (control and safety, health, environment, etc.), and which enables detection by an animal or by an analytical equipment while reducing and possibly eliminating the potential risks for the agent responsible for the control, the detection animal and/or the goods/objects present in the detection area.

### DESCRIPTION OF THE INVENTION

In order to solve these problems, the invention proposes a solution that is simple to use, fast, reliable and involves a low initial investment for implementation thereof. The proposed solution consists of a dynamic collection of actual odorous molecules that are characteristic of the product of interest.

In the context of the invention, by “actual odorous molecule”, it should be understood a molecule emitted by a substance and which can be perceived by the olfactory sense. Preferably, it consists of an organic molecule, generally volatile, with a low molecular mass, in practice less than 350 g/mol, which allows it to vaporize at room temperature so that at least one fraction is found in the gaseous atmosphere surrounding the product/material from which this odorous molecule comes. Furthermore, these molecules can easily diffuse throughout fabrics or plastic films.

Thus, according to a first aspect, the invention relates to a dynamic method for detecting real odorous molecules of interest contained in the atmosphere or in direct proximity to the atmosphere present in an enclosure comprising the following steps:
a) sucking up a sample of the atmosphere of said enclosure by means of an extraction device;
b) making said sample circulate throughout and/or over a collection device capable of adsorbing then absorbing the actual odorous molecules of interest, said device being in the form of a hollow tube open at each of its ends; and
c) having said device analyzed to determine whether the enclosure contains or has contained products of interest.

The method according to the invention is a dynamic method for extracting, collecting and detecting actual odorous molecules. In other words, this is not a static collection device that remains in contact with or proximate to a source of actual odorous molecules in order to collect them as described in the document WO 2018/206808.

According to the invention, by “in direct proximity to the atmosphere present in an enclosure”, it should be understood a short distance, advantageously an area located at less than 2 meters, and possibly at less than 1 meter 50, at less than 1 meter, at less 50 centimeters and possibly at less than 30 centimeters.

According to an essential feature, the device is in the form of a hollow tube open at each of its ends so that the air containing the odorous molecules could pass around, but also inside the tube, which increases the absorption surface.

Thus, a sufficient amount of odorous molecules can be absorbed as of the first passage of air and it is not necessary to provide a recirculation system. The extraction device according to the invention sucks up the air continuously, without it being necessary to establish successive suction, recirculation and then ejection cycles. Hence, the method and the device according to the invention are simpler and more effective.

Furthermore, because air can easily flow through the hollow tube, the used air flow rate may be higher. There is less pressure drop and the risk of obstruction is eliminated. Hence, it is possible to contend with a simpler, compact and inexpensive suction means, such as a domestic vacuum cleaner for example.

According to a particular embodiment, the suction of step a) may be carried out in a continuous or discontinuous mode.

According to a particular embodiment, the circulation of the sample of step b) is carried out continuously, i.e. the sample is sucked up then circulates immediately and without delay throughout the collection device.

According to another embodiment, step b) is carried out discontinuously, i.e. the sample is sucked up then stored in a reservoir. Afterwards, it is blown throughout or onto the collection device.

According to the invention, the analysis of step c) may be carried out by a detection animal and/or an analysis machine. For example, the detection animal is a dog, a rodent such as a rat or even insects such as bees. Advantageously the detection animal is a dog. The animal, in particular the dog, has the advantage of identifying the origin of the molecules of interest in a fraction of a second, unlike an analysis machine. As regards the analytical machines, the most common technique is gas chromatography coupled with a mass spectrometer. For this purpose, it is necessary to collect the odor in order to introduce it into the measuring device and that being so, on a small support compatible with the equipment.

According to the invention, the extraction and collection device is placed in the (direct or close) presence of the atmosphere containing or having contained a source of actual odorous molecules in order to absorb said odorous molecules and is then definitively removed from the source of odorous molecules before being subjected to the analysis step (step c)) of the method of the invention.

For the purposes of the invention, by “atmosphere”, it should be understood air, oxygen, hydrogen, nitrogen, etc.

According to a particular embodiment, the sample of the atmosphere present in or proximate to the enclosure according to the invention represents at least 2.5% of the volume of said enclosure, advantageously at least 5%, 10%, 15%, 20% or even 25%

According to a particular embodiment, the suction volume flow rate of the method of the invention is determined as a function of the volume of the sample. According to the invention, the higher the suction volume flow rate, the larger the amount of actual odorous molecules sucked up by the extraction/collection device of the invention. In other words, the greater the fluid flow, the greater the flux/flow of fluid passing throughout the collection device capable of absorbing the actual odorous molecules of interest and therefore, a larger volume of fluid comes into contact with said collection device ensuring that a large amount of actual odorous molecules are absorbed by said device.

Thus, the method of the invention makes it possible to pass a fluid containing actual odorous molecules of interest with an accurate and specific flow rate over the collection system so that a maximum amount of these odorous molecules are absorbed.

According to a particular embodiment, the actual odorous molecules of interest according to the invention come from a source of odorous molecules selected from the group including narcotics, explosives, weapons, ammunition, bank notes, mold, living organisms such as insects, parasites, bacteria, fungi or viruses; so-called contraband products such as drugs, cigarettes, protected animals or parts of protected animals.

According to a particular embodiment, the enclosure according to the invention is cargo; advantageously a container, a trailer or a pallet, possibly refrigerated or frozen; a room or a building, the interior or exterior of a vehicle. For example, the cargo according to the invention may be that of a boat, a plane, a truck or even a train.

According to another aspect, the invention relates to an extraction device for implementing the method as described before, comprising:
a means for sucking up the sample;
a collection device capable of absorbing the actual odorous molecules of interest, said device being in the form of a hollow tube open at each of its ends; and
means for fastening the collection device to the suction means.

According to a particular embodiment, the sample suction means is a professional or domestic vacuum cleaner or a pump having an air flow rate advantageously comprised between 0.1 and 10 m³/h, preferably in the range of 4 m³/h.

According to the invention, the higher this flow rate, the higher the capacity of the vacuum cleaner to transport air from the suction head to the tank.

This particular embodiment involves the purchase of a professional or domestic vacuum cleaner whose cost is much lower than that of other suction systems, in particular those used conventionally. Furthermore, a professional or domestic vacuum cleaner is readily available and is completely compatible with the device of the invention. The invention also enables the user to work with a battery-powered vacuum cleaner, thereby having a light, small-size and very handy tool.

According to the invention, the collection device capable of absorbing actual odorous molecules is a device for collecting, absorbing (or impregnating) and releasing these molecules over a long period. This collection device ensures the storage of odorous molecules for later use, which limits the risks for the agent responsible for control and the animal since they are never in direct contact with the source of odorous molecules, or with the atmosphere of the enclosure.

Depending on the hazardousness of the molecules to be detected and the enclosures likely to contain them, the movement of the extraction device of the invention is automated to avoid any risk for the agent and for the dog. In other words and in a particular embodiment, the invention also relates to a motorized robot which comprises an extraction device as described before.

Thus, the robot which carries the extraction device may, for example, be autonomous, remotely-controlled or pre-programmed to move to the extraction location thanks to its motorization, without the agent having to approach. Once on site, the extraction device is set into operation either directly by the robot, or remotely by the agent or any other system, so that the atmosphere sample is sucked up and the odorous molecules collected. Afterwards, thanks to its motorization, the robot can move to the place where the collection device will be analyzed or up to a place far away from the enclosure where it will be taken care of by the agent.

Such motorized robots, capable of moving autonomously, remotely controlled or pre-programmed up to a given location, are known from the prior art and a person skilled in the art will easily be able to select one, compatible with the extraction device and the method according to the invention, without it being necessary to describe it in more detail.

Hence, the invention makes it possible to quickly collect the odorous molecules present in a large volume and to concentrate them in a collection device capable of absorbing the actual odorous molecules of interest. This device capable of absorbing the odorous molecules is capable of storing them in large amounts, then releasing them in a controlled manner over a long period.

According to a particular embodiment, the collection device capable of absorbing the actual odorous molecules according to the invention is olfactory neutral.

According to a particular embodiment, the collection device capable of absorbing the actual odorous molecules according to the invention comprises, advantageously consists of, a polymer or a mixture of polymers enabling absorption of the odorous molecules of interest in the mesh of its polymeric network. Thus, the actual odorous molecules are not simply adsorbed but absorbed into the device. Thus, a large amount of odorous molecules can be stored and then released during use of the device, in particular for step c) of the method according to the invention. Advantageously, the restitution of the odorous molecules according to the invention does not require a step of desorption of said device other than natural desorption when the detection is carried out by an animal such as a dog for example.

The structure of the polymer(s) used in the invention is different from the structure of the odorous molecules of interest to be collected, in particular with the aim of not inducing bias in the analysis step of the method of the invention (step c)). Furthermore, said structure of the polymer(s) is chemically compatible with the molecule of interest so that absorption could occur therein. Hence, the odorous molecule of interest is at least partially soluble in the polymer(s) constituting the device so that it can be absorbed and stored there passively.

According to a particular embodiment, the collection device capable of absorbing the actual odorous molecules according to the invention is not pre-impregnated with a natural or synthetic odor (pseudo-odor) and it does not contain particles of actual material from the source of odorous molecules.

According to a particular embodiment, the collection device capable of absorbing the actual odorous molecules according to the invention comprises, advantageously consists of, a pure polymer or a mixture of polymers, additives may possibly be added. This material has a high capacity for absorption of odorous molecules of different natures, whether polar or non-polar, and in particular VOCs (volatile organic compounds). It also has the capacity to release the odorous molecules it contains in a controlled manner in terms of flow rate and duration. Finally, it is easy to implement by conventional processes for implementing polymers, namely and without limitation: extrusion, spinning, injection, pressing, casting, rotational molding, extrusion blow molding, stretch blow molding, extrusion inflation, thermoforming, compression molding, etc.

According to a particular embodiment, in the case where the collection device capable of absorbing the real odorous molecules according to the invention comprises, advantageously consists of, a mixture of polymers, if these are not miscible then they are mixed in relative proportions allowing a co-continuous polymer network to be obtained. In this context, the polymers of the mixture are present at the surface to be in contact with the odorous molecules to be captured during absorption. Hence, each type of polymer forming a continuous network, the odorous molecules can be stored by diffusion throughout the volume of the material of the device according to the invention.

According to a particular embodiment, the polymer(s) used to make a device according to the invention are selected from the group consisting of natural, artificial or synthetic polymers, advantageously artificial polymers. Alternatively, it may consist of a mixture between at least two polymers of different natures (for example natural polymer with synthetic polymer). Among the natural polymers capable of being implemented in the invention, mention may be made of polysaccharides, glycosamininoglycans, lignin, nucleic acids, scleroproteins, natural rubber and polyhydroxyalkanoates. Among the artificial polymers capable of being implemented in the invention, mention may be made of cellulose acetate and nitrocellulose. Among the synthetic polymers capable of being implemented in the invention, mention may be made of polyolefins, vinyl polymers, styrenic polymers, polyesters, polyamides, polyurethanes, polycarbonates, acrylic polymers, aminoplast and phenoplast polymers, polyacetals, silicone polymers, polyimides, halogenated polymers, polydimethylsiloxanes, epoxies, thermostable polymers, elastomers and electroactive polymers.

According to the present invention, the device for collecting and rendering actual odorous molecules of interest comprises, consists of, a polymer or a mixture of polymers, advantageously having an olfactory neutrality, selected from the group consisting of polyether-block-amide (PEBA), polypropylene (PP), polyolefins, a mixture of polyether-block-amide with one or more silicone-type polymer(s) (PDMS), and aromatic polymers.

According to a particular embodiment, the polymer according to the invention is selected from the group consisting of polyether-block-amide (or PEBA), a mixture of polyether-block-amide with one or more polyolefin-type polymer(s) (polyethylene or polypropylene), a mixture of polyether-block-amide with one or more silicone-type polymer(s) (PDMS), a mixture of polyether-block-amide with one or more aromatic polymer(s), for example and without limitation a polystyrene or a mixture of polyether-block-amide with one or more polymer(s). More generally, the polymer added to PEBA is selected from among those making it possible to absorb and then release one or more application-specific odorous molecule(s).

According to a particular embodiment, the polymer is PEBA alone or in combination with another polymer.

According to a particular embodiment, the collection device which is in the form of a hollow tube open at both of its ends, is advantageously made of PEBA or made of one or a mixture of the aforementioned polymers.

According to a particular embodiment, the means for fastening the collection means to the suction means according to the invention is in the form of a sleeve, preferably with a tubular general shape, comprising:
a wall;
a first open end capable of sucking up the atmosphere sample;
a second end, preferably opposite to the first end capable of being connected to the suction means; and
a means for retaining the collection means.

According to a particular embodiment, the open end of the sleeve has at least one means for connection to an extension, said extension being for example, of the flexible tube type with a variable diameter depending on the applications and which can be easily inserted into freight cargo or into a room, for example through the gap in a door, throughout a keyhole, etc.

For example, the at least one connection means is in the form of a tapping or a thread intended to cooperate with a corresponding means provided on the extension.

To enable the other end of the sleeve to be connected to the suction means, this also has at least one connection means, preferably a clip-on one. For example, these may consist of male/female elements intended to cooperate with corresponding elements of the suction means.

According to the invention, the sleeve is provided with a means for retaining the collection device for example in the form of a housing intended to receive said collection device. Advantageously, the housing has a frustoconical tubular general shape to enable the collection device, in practice the tube, to be effectively fastened by insertion.

According to a particular embodiment, the wall of the housing includes at least one recess, so as to enable the sucked atmosphere to be, in particular, in direct contact with the external surface of the collection device held in the housing.

According to the invention, the collection device is easily removed from the housing and therefore from the extraction device to be analyzed afterwards (for example: detection dog, GC/MS, etc.) so as to confirm the presence, or not, current or past, of products of interest in the enclosure subject to inspection.

In practice, the collection device has to be handled to be inserted into and extracted from the housing. This handling is performed manually, in particular by an agent wearing single-use gloves or without direct handling, i.e. using a tool, for example pliers.

In practice, the means for retaining the collection device is maintained, advantageously at the center of the sleeve, by means of at least one means for connection to the wall of said sleeve, for example in the form of one or more fins.

According to a particular embodiment, the sleeve contains 4 fins placed at 90° with respect to each other.

According to a particular embodiment, the extraction device according to the invention is transportable. For example, said device can be transported by an agent responsible for control, an animal, or even a motorized vehicle such as a robot.

Advantageously, the extraction device according to the invention can be activated/deactivated manually and/or remotely by remote control.

According to a particular embodiment, the extraction device is made of stainless steel and/or plastic.

It appears from the above that the extraction device according to the invention has the advantages of:
having a shape ensuring a minimum pressure drop in order to guarantee a high suction flow rate;
be as light as possible (presence of recess and light manufacturing materials) and therefore easy to transport and to handle;
consist of durable and standard materials, in particular in the medical industry (for example 316L stainless steel).

Furthermore and unlike the filtration collection device of the prior art which aims to collect suspended particles directly emitted by the product of interest, the invention has the following advantages of:
collecting the odorous molecules which form the olfactory signature of the product of interest;
being able to detect elements of interest (products, animals) which do not emit particles into the air (via a natural process or because of their packaging); and
being able to detect the past presence of elements of interest.

According to another aspect, the invention relates to a means for fastening a collection device capable of absorbing the real odorous molecules of interest intended to be connected to the previously-described suction means of the device. It is characterized in that it comprises a sleeve comprising:
a wall;
a first open end capable of sucking up the atmosphere sample;
a second end capable of being connected to the suction means; and
a means for retaining the collection device.

According to another aspect, the invention relates to the use of the extraction device as described before to control the legality and/or ensure the safety and/or ensure the security of a cargo, a room, a building or a vehicle.

According to a particular embodiment, the use of the device according to the invention is carried out in at least one of the following fields: customs control, search of vehicles, searches in the context of police/gendarmerie investigations, control of the presence of products of interest in buildings, detection of undesirable organisms (insects, for example bedbugs or micro-organisms) in individuals, professionals receiving the public (hotels) or in health establishments (hospitals, etc.).

Thus, the present invention:
allows performing faster detections (or checks) thanks to a higher air flow since the extraction device of the invention ensures less pressure drop thanks to the absence of filter (reduction in cost/control);
is more reliable thanks to a technology based on the absorption of odorous molecules which are easier to collect than particles and more representative of the actual odor of the product of interest (increase in seizures and therefore reduction in loss of income for States);
is universal since all types of cargo can be controlled, even buildings or vehicles;
guarantees better security for control agents, detection animals or the goods;
is implemented with a low initial investment (because the device adapts to commercial suction systems);
uses already available resources (customs officers, detection dog);
is more discreet in that it is not always necessary to break the seals to perform a check, the suction tube being able to slip, for example, under the tarpaulin of a truck; and
is effective since the device of the invention is easy to handle, small in size, low in weight, gives the possibility of working on battery power and therefore in places without an electrical connection.

### BRIEF DESCRIPTION OF THE FIGURES

The manner of carrying out the invention, as well as the advantages which result therefrom, will emerge clearly from the description of the embodiments that follow, with reference to the appended figures wherein:

FIG. 1 is a schematic sectional view of the extraction device of the invention.

FIG. 2 is a schematic top view of the sleeve of the extraction device.

FIG. 3 is a cross-section of FIG. 2 according to the plane AA.

FIG. 4 is a perspective view of the sleeve in the absence of the collection device.

FIG. 5 is a schematic sectional view of the extraction device of the invention.

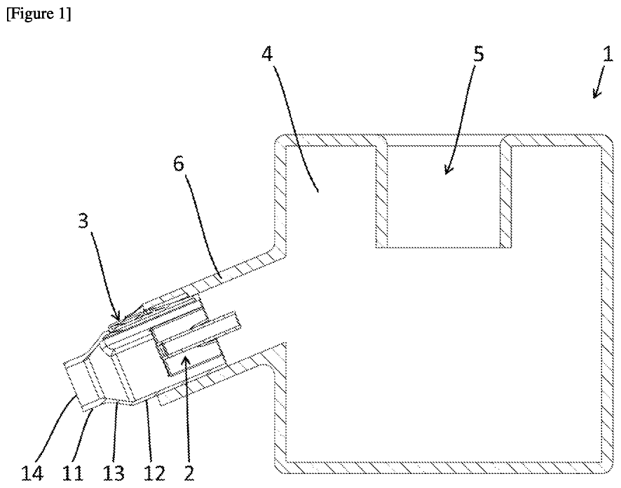

### DESCRIPTION OF THE EMBODIMENTS

The device of the invention essentially comprises three elements which are a suction means (1), a collection device (2) capable of absorbing the odorous molecules and a means for fastening (3) the collection device (2) to the suction means (1).

The suction means (1) essentially comprises a dust receiving tank (4), a motor (5) and an adapter (6) to the fastening means (3).

In practice, the suction means is a domestic vacuum cleaner, for example a DYSON® brand vacuum cleaner, to which the fastening means (3) is connected instead of the standard modules found on the market.

A vacuum cleaner can be used with an air flow rate of about 4 m³/h

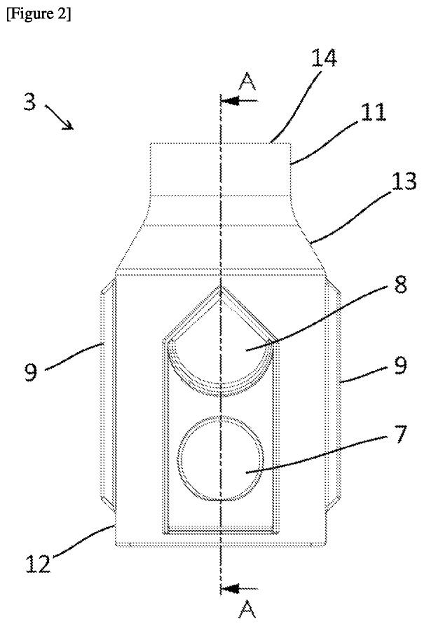

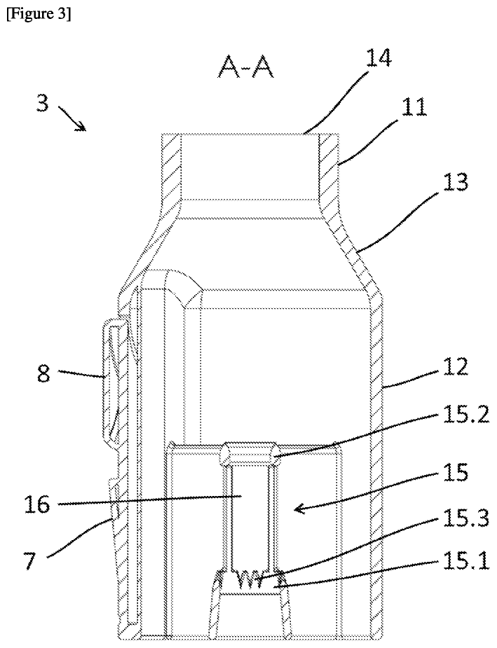

The adapter (6) is intended to be connected to the fastening means (3) of the collection device (2), in particular by clipping. More particularly, clipping means are shown in FIGS. 2 to 4. In this embodiment, these consist of two buttons (7, 8) intended to cooperate with corresponding openings arranged on the adapter (6) of the vacuum cleaner.

Still in this particular embodiment, the two buttons (7,8) are lined with ribs (9) intended to cooperate with corresponding grooves in the sleeve (6) which are not shown thereby making it possible to avoid any play between the two elements.

More particularly, the collection device (2) is shown in FIG. 5). It is in the form of a hollow tube (10) whose two ends (10.1, 10.2) are open so as to allow the sucked atmosphere sample to pass through. In this embodiment, the tube is made of PEBA but can be manufactured of all the materials as mentioned before.

The extraction device according to the invention comprises a third element which is the fastening means (3) of the collection device (2).

This fastening means is shown in FIGS. 2 to 4.

It is in the form of a sleeve with a tubular general shape, having three distinct sections in the direction of extraction of the atmosphere sample respectively a first tubular section (11), a second tubular section (12) with a diameter larger than that of the first section (11), the two sections (11, 12) being connected by a conical section (13). This general shape of the sleeve makes it possible to optimize the dynamics of extraction of the atmosphere sample and the flow of the fluid in said sleeve throughout the hollow tube (10) as well as at its external surface.

In some cases, the open end (14) of the sleeve may be provided with means for connection to an extension which is not illustrated. In practice, the extension is in the form of a flexible tube and intended to be connected to the wall of the cargo, such as for example the wall of a container.

The sleeve (3) further includes a retaining means (15) of the collection device (2) itself having a frustoconical tubular general shape so as to receive the collection device by insertion. The retaining means is positioned at the center of the sleeve in the longitudinal direction and thus has 2 rings respectively a first ring (15.1) with a diameter slightly larger than that of the collecting tube and a second ring (15.2) with a diameter substantially equal to that of the collecting tube. The first ring (15.1) is further provided on its periphery opposite the second ring (15.2) with teeth (15.3) oriented in the direction of the second ring. Thus, these teeth make it possible to hold the collecting tube in position in its housing.

The wall of the housing (15) further has recesses in the form of apertures (16) enabling the surface of the tube to be in direct contact with the atmosphere sample entering by suction. In other words, most of the inner and outer surfaces of the tube are in contact with the extracted atmosphere sample.

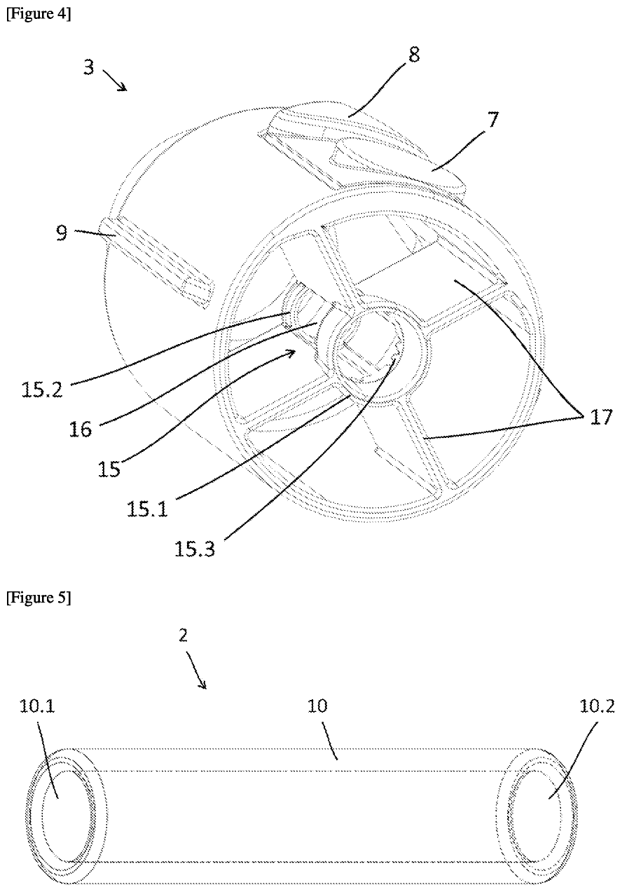

As shown in FIG. 4, the housing (15) is connected to the wall of the sleeve (3) by means of fins (17) which are 4 in number in the embodiment as shown. The fins are distributed symmetrically all around the sleeve.

In practice, and as explained before, the collection tube is received in an odor-sealed packaging. The operator then simply, after opening the blister, inserts the collecting tube into the housing (15) provided to this end, by insertion, possibly using a tool such as pliers. Afterwards, the sleeve is connected to the suction device (1) via the adapter (6). The operator then simply activates the vacuum cleaner and sucks up a sample of the atmosphere to be tested proximate the container or possibly directly into the container, in particular via an extension.

In practice, the volume of the sample is determined according to the nature of the products likely to be identified and/or the volume of the enclosure in which said products are potentially present. For example, this volume may range from 2.5% to 10%.

The suction operation is very quick, around a few minutes. Once the suction has been carried out, the operator simply grasps the tube of the retaining means (15) and presents it, immediately or not, to a detection dog. The PEBA constituting the tube allows odors to be fixed for a long time.

It will then only take a few seconds for the dog to identify the odor and therefore relate it to a specific material or product.

The invention and the advantages which result therefrom emerge clearly from the preceding description: we note in particular the ease of use of the device of the invention which, combined with the collection device, makes it possible to identify most illicit substances and in particular in the context of freight control.

1. A dynamic method for detecting actual odorous molecules of interest contained in the atmosphere or in direct proximity to the atmosphere present in an enclosure comprising the following steps:
a) Absorbing a sample of the atmosphere present in or proximate to said enclosure by means of an extraction device;
b) Making said sample circulate/pass through and/or over a collection device capable of absorbing the actual odorous molecules of interest, said device being in the form of a hollow tube open at each of its ends; and
c) Having said device analyzed by a detection animal and/or an analysis machine.

2. The method according to claim 1, characterized in that the actual odorous molecules of interest come from a source of odorous molecules selected from the group including narcotics, explosives, weapons, ammunition, bank notes, mold, living organisms such as insects, parasites, bacteria, fungi or viruses and so-called contraband products such as drugs, cigarettes, protected animals or parts of protected animals.

3. The method according to claim 1, characterized in that the enclosure is cargo; advantageously a container, a trailer or a pallet, possibly refrigerated or frozen; a room, a building or a vehicle.

4. An extraction device for implementing the method according to claim 1, comprising:
a suction means for collecting the sample;
a collection device capable of absorbing the actual odorous molecules of interest, the device being in the form of a hollow tube open at each of its ends; and
a means for fastening the collection device to the suction means.

5. The extraction device according to claim 4, characterized in that the collection device consists of a polymer selected from the group consisting of polyether-block-amide (or PEBA), a mixture of polyether-block-amide with one or more polyolefin-type polymer(s) (polyethylene or polypropylene), a mixture of polyether-block-amide with one or more silicone-type polymer(s) (PDMS), a mixture of polyether-block-amide with one or more aromatic polymer(s).

6. The extraction device according to claim 4, characterized in that the fastening means is in the form of a sleeve comprising:
a wall;
a first open end capable of sucking in the atmosphere sample;
a second end capable of being connected to the suction means; and
a retaining means of the collection device.

7. The extraction device according to claim 6, characterized in that the first end has means for connection to an extension, of a flexible tube type.

8. The extraction device according to claim 6, characterized in that the second end has at least one clip-on connection means, of a male/female element type intended to cooperate with at least one corresponding element of the suction means.

9. The extraction device according to claim 6, characterized in that the retaining means is in the form of a generally frustoconical tubular housing capable of fastening the collection device by insertion.

10. The extraction device according to claim 9, characterized in that housing has a wall that includes at least one recess.

11. The extraction device according to claim 6, characterized in that the retaining means is held at the center of the sleeve by means of connecting means to the wall of said sleeve, for example in the form of fins.

12. The extraction device according to claim 4, characterized in that it is transportable.

13. A motorized robot characterized in that it comprises an extraction device according to claim 4.
